(19) United States
(12) Patent Application Publication
(10) Pub. No.: US 2025/0107701 A1
(43) Pub. Date: Apr. 3, 2025

(54) STEREOSCOPIC ENDOSCOPE CAMERA TOOL DEPTH ESTIMATION AND POINT CLOUD GENERATION FOR PATIENT ANATOMY POSITIONAL REGISTRATION DURING LUNG NAVIGATION

(71) Applicant: Covidien LP, Mansfield, MA (US)

(72) Inventors: Vaclav Grym, Eagan, MN (US); Guy Alexandroni, Yehud-Monosson (IL); Jonathan Scott Thomson, Stoneham, MA (US); Daniel Ovadia, Ramat Gan (IL); Shaked Pessah, Maccabim-Reut (IL); Nathan J. Knutson, Long Lake, MN (US); Darrick Chekas, Cheshire, CT (US); Badr Elmaanaoui, Belmont, MA (US)

(73) Assignee: Covidien LP, Mansfield, MA (US)

(21) Appl. No.: 18/896,859

(22) Filed: Sep. 25, 2024

Related U.S. Application Data
(60) Provisional application No. 63/541,898, filed on Oct. 1, 2023.

(51) Int. Cl. A61B 1/00; A61B 1/267; G06T 7/33; G06T 7/593

(52) U.S. Cl. CPC A61B 1/00193; A61B 1/00097; A61B 1/2676; G06T 7/344; G06T 7/593; A61B 1/00114; G06T 2207/10028; G06T 2207/10068; G06T 2207/20084; G06T 2207/30061

(57) ABSTRACT

Endoscopic systems and methods use a multi-view camera tool inside the body, including airways of a lung, to capture images, and use a combination of positional informational from EM and/or IMU sensors of the multi-view camera tool to estimate image depth and generate a three-dimensional (3D) point cloud volume of the patient anatomy. The 3D point cloud volume is generated from a known vantage point using stereoscopic images captured by the camera tool. This 3D structure generation may use a stereo image rectification algorithm. A machine learning algorithm may also be applied in place of, or in combination with, an image rectification algorithm to improve computational efficiency.

### CROSS-REFERENCE TO RELATED APPLICATIONS

This application claims the benefit of the filing date of provisional U.S. Patent Application No. 63/541,898, filed Oct. 1, 2023.

### FIELD

The technology of the disclosure is generally related to a stereoscopic endoscope camera tools and methods of using the camera tools to estimate image depth and generate a point cloud for registering patient anatomical positional information to images from a different imaging modality during lung navigation.

### BACKGROUND

When performing a medical procedure, clinicians often rely on patient data including X-ray data, computed tomography (CT) scan data, magnetic resonance imaging (MRI) data, or other imaging data that allows the clinician to view the internal anatomy of a patient. The imaging data is also utilized to identify targets of interest and to develop strategies for accessing the targets of interest for surgical treatment. Further, the imaging data has been used to create a three-dimensional (3D) model of the patient's body to guide navigation of the medical device to a target of interest within a patient's body.

Small discrepancies between the actual location and an estimated location of the medical device may cause undesired consequences in the medical procedure. Thus, precision in estimating the actual location of the medical device with a sufficient level of accuracy is highly desirable during medical procedures.

### SUMMARY

The techniques of this disclosure generally relate to stereoscopic endoscope camera and sensor tools and methods of using the camera and sensor tools to estimate image depth and generate a point cloud for registering patient anatomical positional information to images from a different imaging modality during lung navigation.

In one aspect, the present disclosure provides a camera and sensor tool. The camera and sensor tool includes a sensor pack at a distal end portion of the camera and sensor tool. The sensor pack includes a structural member, a cable assembly, one or more cameras, an electromagnetic (EM) sensor assembly, an inertial measurement unit (IMU), an illumination source, and one or more lenses.

The one or more cameras are coupled to the structural member and electrically coupled to the cable assembly. The electromagnetic (EM) sensor assembly is coupled to the structural member and electrically coupled to the cable assembly. The inertial measurement unit (IMU) is coupled to the structural member and electrically coupled to the cable assembly. The one or more lenses are optically coupled to apertures of the one or more cameras.

Implementations of the camera and sensor tool may include one or more of the following features. In aspects, the structural member may be a length of flat wire or rigid wire. In aspects, the sensor pack and the cable assembly may be encased within a sheath.

In another aspect, the present disclosure provides a method of registering stereo images of at least one body lumen to a three-dimensional (3D) model. The method includes illuminating, by a camera and sensor tool disposed within an endoscopic catheter, a feature of the at least one body lumen; capturing, by one or more cameras of the camera and sensor tool, stereoscopic images of the feature of the at least one body lumen; and matching points between the stereoscopic images, yielding matched points. The method also includes estimating depth information based on the matched points, converting the depth information to a point cloud volume based on intrinsic parameters of the one or more cameras, and registering the point cloud volume to the 3D model of the at least one body lumen.

Implementations of the method may include one or more of the following features. In aspects, the at least one body lumen may be an airway of a lung. In aspects, the method also includes selectively illuminating the feature of the at least one body lumen with different illumination sources of the camera and sensor tool.

In aspects, capturing the stereoscopic images of the feature of the at least one body lumen may include capturing the stereoscopic images of the feature of the at least one body lumen through a pair of lenses adjacent to an aperture of a camera of the one or more cameras.

In aspects, estimating the depth information may include estimating the depth information using a neural network.

In aspects, the method may include generating the 3D model based on preoperative radiographic images of the at least one body lumen.

In aspects, the at least one body lumen forms at least a portion of a bronchial tree.

In aspects, the method may include rectifying the stereoscopic images before matching points between the stereoscopic images. Rectifying the stereoscopic images may include applying an image rectification algorithm to the stereoscopic images. The image rectification algorithm may be at least one of an epipolar rectification algorithm, Hartley's rectification algorithm, a polar rectification algorithm, a recursive rectification algorithm, a 3D rotation rectification algorithm, a non-parametric rectification algorithm, a shear-based rectification algorithm, or an automatic rectification algorithm.

In another aspect, the present disclosure provides a system. The system includes a catheter, a sensor pack at a distal end portion of the catheter, a processor, and memory. The sensor pack includes a structural member, a cable assembly, one or more cameras, an illumination source, and one or more lenses. The one or more cameras are coupled to the structural member and electrically coupled to the cable assembly. The one or more lenses are optically coupled to apertures of the one or more cameras.

The memory has stored thereon instructions, which when executed by the processor, causes the processor to: illuminate, by the illumination source, a feature of the at least one body lumen; capture, by the one or more cameras, stereoscopic images of the feature of the at least one body lumen; match points between the stereoscopic images, yielding matched points; estimate depth information based on the matched points; convert the depth information to a point cloud volume based on intrinsic parameters of the one or more cameras; and register the point cloud volume to the 3D model of the at least one body lumen.

Implementations of the system may include one or more of the following features. In aspects, the structural member may be a length of flat wire or rigid wire. In aspects, the sensor pack and the cable assembly may be encased within a sheath.

In aspects, the sensor pack may include an electromagnetic (EM) sensor assembly coupled to the structural member and electrically coupled to the cable assembly.

In aspects, the sensor pack may include an inertial measurement unit (IMU) coupled to the structural member and electrically coupled to the cable assembly.

In aspects, capturing the stereoscopic images of the feature of the at least one body lumen may include capturing the stereoscopic images of the feature of the at least one body lumen through a pair of lenses adjacent to an aperture of a camera of the one or more cameras. In aspects, estimating the depth information may include estimating the depth information using a neural network.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF DRAWINGS

FIG. 1 is a block diagram that illustrates a system for acquiring and processing 3D CBCT scans of a patient and a location board;

FIG. 2 is a circuit block diagram that illustrates the workstation of the system of FIG. 1;

FIG. 3 is a block diagram that illustrates a camera and sensor tool for use in a catheter or working channel;

FIG. 4 is a side view of a camera and sensor tool;

FIG. 5 is a side view of another example of a camera and sensor tool;

FIG. 6 is a perspective view of a distal end portion of the other example of the camera and sensor tool of FIG. 5;

FIG. 7 is a top view of the distal end portion and length portions of the other example of the camera and sensor tool of FIG. 5;

FIGS. 8A and 8B are block diagrams that illustrate examples of dual camera configurations;

FIG. 9 is a block diagram that illustrates an example of a camera module including staggered cameras and stereoscopic lenses;

FIG. 10 shows block diagrams that illustrate examples of camera modules including a single camera and stereoscopic lenses;

FIG. 11 shows block diagrams that illustrate an example of a camera module including a single camera and an adaptive lens;

FIG. 12 is a block diagram that illustrates articulation of a camera tool to increase the field of view of the camera; and

FIG. 13 is a flowchart of an example of a method of generating a point cloud volume from stereoscopic images of at least one body lumen acquired by a camera and sensor tool of the disclosure and registering the point cloud volume to a three-dimensional (3D) model.

### DETAILED DESCRIPTION

Successful navigation of an endoscopic catheter through a patient's lung is dependent upon an accurate measurement of position within a patient's lung anatomy. Patient anatomy can be successfully mapped to a digital model using 3D imaging techniques, such as CT imaging. A position within a 3D volume can be calculated with a variety of methods, including signal demodulation of a generated electromagnetic (EM) field and relative motion calculated from acceleration sensed by an inertial measurement unit (IMU). Combining the catheter position information within a volume and the modeled patient anatomy requires a registration of the two known coordinate systems.

This disclosure uses a multi-view (e.g., dual-view) endoscopic camera tool inside the body, e.g., the lungs, and uses a combination of positional information and still or video images captured by the camera tool to estimate the image depth and generate a 3-dimensional point cloud of the patient anatomy based on the combination of positional information and still or video images. The 3D point cloud volume is generated from a known vantage point using stereoscopic images captured by the multi-view endoscopic camera tool. This disclosure leverages 3D structure generation using a stereo image rectification algorithm, which is an application of stereo vision. A machine learning algorithm may also be applied in place of, or in combination with, any of the image rectification algorithms described herein to improve computational efficiency.

According to aspects, stereoscopic images may be obtained with either two cameras offset a known distance and positioned to capture an overlapping field, one camera with a specially designed lens setup, or one camera with the ability to mechanically change the view position. In particular, the following camera module 321 configurations may be used to obtain stereoscopic views:
Dual camera positioning, by which cameras are positioned opposite or orthogonal to each other.
Lens configurations for a staggered camera configuration.
Lens configurations for stereoscopic vision with a single camera.
Adaptive lens for focal point adjustment to generate a stereoscopic view with a single camera.
Mechanical positioning of a catheter to create a stereoscopic or swept view.

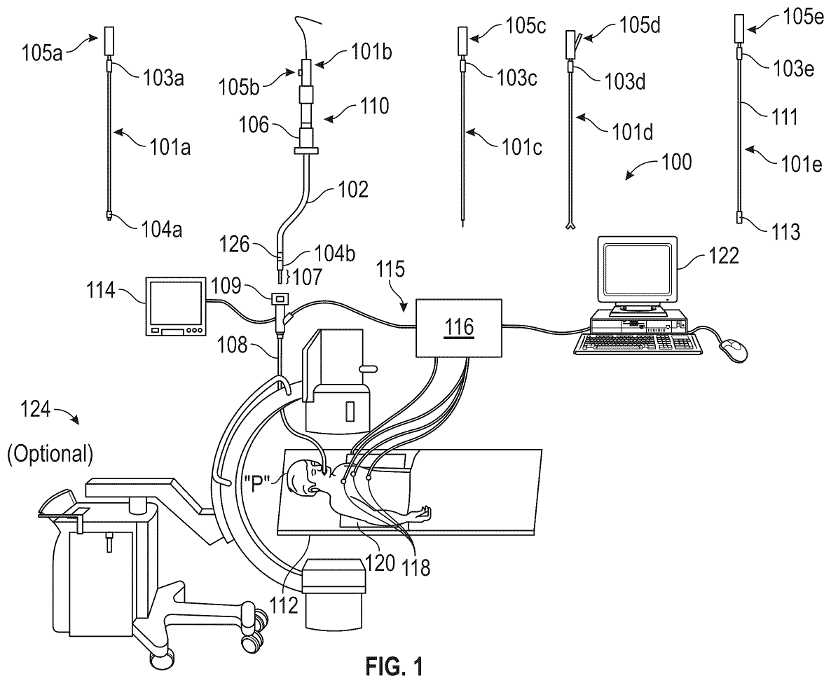

FIG. 1 is a perspective view of an example of a system 100 for facilitating navigation of a medical tool, e.g., a catheter or a camera and sensor tool 101e, to a target or target area via airways of the lungs. During navigation, the system 100 may be configured to generate a point cloud volume from stereoscopic images of at least one body lumen acquired by the camera and sensor tool 101e and registering the point cloud volume to a three-dimensional (3D) model generated, for example, from preoperative radiographic images. The system 100 may also be configured to apply a machine learning-based stereoscopic image rectification algorithm to stereoscopic images acquired by the camera and sensor tool 101e to facilitate point matching in the process of generating a point cloud volume.

The system 100 may be further configured to construct radiographic-based volumetric data of a target area from intraprocedural 2D radiographic images, e.g., intraprocedural fluoroscopic and/or CBCT images, to confirm navigation of a navigation catheter 102, e.g., an extended working channel (EWC) or a smart extended working channel (sEWC), to a desired location near the target area, where a tool, e.g., a camera and sensor tool 101e, may be placed through and extending out of the navigation catheter 102. In aspects, the imaging system 124 of the system 100 may include one or more of a C-arm fluoroscope, a 3D cone-beam computed tomography (CBCT) imaging system, and a 3D fluoroscopic imaging system.

The system 100 may be further configured to facilitate approach of a medical tool to the target area and to determine the location of the medical tool with respect to the target by using electromagnetic navigation (EMN) of the navigation catheter 102. One such EMN system is the ILLUMISITE system currently sold by Medtronic PLC, though other systems for intraluminal navigation are considered within the scope of this disclosure.

One aspect of the system 100 is a software component for reviewing computed tomography (CT) image scan data that has been acquired separately from the system 100. The review of the CT image data allows a user to identify one or more targets, plan a pathway to an identified target (planning phase), navigate the navigation catheter 102 (e.g., and EWC or sEWC) including the a camera and sensor tool 101e to the target (navigation phase) using a user interface running on a computer system 122, and confirming placement of a distal end portion of the navigation catheter 102 near the target using the camera and sensor tool 101e and/or using one or more electromagnetic (EM) sensors 104b, 126 disposed in or on the navigation catheter 102 at a predetermined position at or near the distal end portion of the navigation catheter 102. The target may be tissue of interest identified by review of the CT image data during the planning phase.

Following navigation of the navigation catheter 102 near the target, a medical tool, such as a biopsy tool, an access tool, or a therapy tool, e.g., a flexible microwave ablation catheter, may be inserted into and may be fixed in place with respect to the navigation catheter 102 such that a distal end portion of the medical tool extends a desired distance 107 beyond the distal end of the navigation catheter 102 and the navigation catheter 102 is further navigated using EM navigation to obtain a tissue sample, enable access to a target site, or apply therapy to the target using the medical tool.

As shown in FIG. 1, the navigation catheter 102 is part of a catheter guide assembly 110. In practice, the navigation catheter 102 is inserted into a bronchoscope 108 or other suitable endoscope for access to a luminal network of the patient P. Specifically, the navigation catheter 102 of catheter guide assembly 110 may be inserted into a working channel of the bronchoscope 108 for navigation through a patient's luminal network. A bronchoscope adapter 109 is coupled to the proximal end portion of the bronchoscope. The bronchoscope adapter 109 may be the EDGE™ Bronchoscope Adapter, which is currently marketed and sold by Medtronic PLC. The bronchoscope adapter 109 is configured either to allow motion of the navigation catheter 102 through the working channel of the bronchoscope 108 (which may be referred to as an unlocked state of the bronchoscope adapter 109) or prevent motion of the navigation catheter 102 through the working channel of the bronchoscope (which may be referred to as an unlocked state of the bronchoscope adapter 109). Alternatively, the camera and sensor tool 101e may be inserted into the working channel of the bronchoscope 108 in place of the navigation catheter 102.

Aspects of the disclosure may be applied to a variety of procedures including biopsy, ablation, or marker placement procedures. For example, the procedures may involve one or more of a locatable guide 101a, a microwave ablation tool 101b, a biopsy needle 101c, a forceps 101d, or a camera and sensor tool 101e. The locatable guide (LG) 101a, which may be a catheter, and which may include a sensor 104a similar to the sensor 104b, is inserted into the navigation catheter 102 and locked into position such that the sensor 104a extends a predetermined distance beyond the distal end portion of the navigation catheter 102. The camera and sensor tool 101e of the disclosure includes a sensor pack 113, which may incorporate miniaturized LEDs, and a catheter assembly 111, which may include one or more camera wires 311, one or more EM sensor wires 312, one or more IMU sensor wires 314, one or more pull wires, and/or one or more illumination optical fibers 316 (in aspects that do not incorporate miniaturized LEDs).

The tools 101a-101e may include a fixing member 103a-e such that when the fixing member 103a-103e of the tools 101a-101e engages, e.g., snaps in, with the proximal end portion of the handle 106 of the catheter guide assembly 110, the tools 101a-101e extend a predetermined distance 107 beyond a distal tip or end portion of the navigation catheter 102. The predetermined distance 107 may be based on the length of the navigation catheter 102 and a length between the end portion of the handle 105a-c or the fixing member 103a-103e and the distal end portion of the LG 101a or the other medical tools 101b-101e. In aspects, the handles 105a-105e may include control objects, e.g., a button or a lever, for controlling operation of the medical tools 101a-101c.

In some aspects, the position of the fixing member 105a-105e along the length of the medical tools 101a-101e may be adjustable so that the user can adjust the distance by which the distal end portion of the LG 101a or the medical tools 101b-101e extend beyond the distal end portion of the navigation catheter 102. The position and orientation of the LG sensor 104a or the sensor pack 113 of the camera and sensor tool 101e relative to a reference coordinate system within an electromagnetic field can be derived using an application executed by the computer system 122. In some aspects, the navigation catheter 102 may function as the LG 101a, in which case the LG 101a may not be used. In other aspects, the navigation catheter 102 and the LG 101a may be used together. For example, data from the sensors 104a and 104b may be fused together. Catheter guide assemblies 110 are currently marketed and sold by Medtronic PLC under the brand names SUPERDIMENSION® Procedure Kits, or EDGE™ Procedure Kits, and are contemplated as useable with the disclosure.

The system 100 generally includes an operating table 112 configured to support a patient P; a bronchoscope 108 configured for insertion through patient P's mouth into patient P's airways; monitoring equipment 114 coupled to bronchoscope 108 (e.g., a video display for displaying the video images received from the video imaging system of bronchoscope 108 or the images received from the camera and sensor tool 101c); and a tracking system 115 including a tracking module 116, patient sensor triplet (PST) 118, and a location board 120. The location board 120 includes one or more EM transmitters for generating an EM field. The location board 120 may be in the form of a transmitter mat.

The location board 120 may also include fiducials, which may be embedded or otherwise incorporated into the location board 120, and which are designed and/or arranged to appear in radiographic images for the purpose of creating a 3D reconstruction from the radiographic images. Since the fiducials may be radiographically dense, the fiducials create artifacts on the radiographic images, e.g., intraoperative CBCT images. The system 100 further includes a computer system 122 on which software and/or hardware are used to facilitate identification of a target, planning a pathway to the target, navigating a medical tool to the target, and/or confirmation and/or determination of placement of the navigation catheter 102, or a suitable tool therethrough, relative to the target.

As noted above, an optional imaging system 124 capable of acquiring CBCT images or fluoroscopic images of the patient P is also included in the system 100. The images, sequence of images, or video captured by the imaging system 124 may be stored within the imaging system 124 or transmitted to the computer system 122 for storage, processing, and display. Additionally, the imaging system 124 may move relative to the patient P so that images may be acquired from different angles or perspectives relative to patient P to create a series of CBCT images.

The pose of the imaging system 124 relative to patient P and while capturing the images may be estimated via markers incorporated into the location board 120, the operating table 112, or a pad (not shown) placed between the patient and the operating table 112. The markers are positioned under patient P, between patient P and operating table 112 and between patient P and a radiation source or a sensing unit of the imaging system 124. The markers may have a symmetrical spacing or may have an asymmetrical spacing, a repeating pattern, or no pattern at all. The imaging system 124 may include a single imaging system or more than one imaging system. When a CBCT system is employed, the captured images can be employed to confirm the location of the navigation catheter 102 and/or one of the medical tools 101a-101e within the patient, update CT-based 3D modeling, or replace pre-procedural 3D modeling with intraprocedural modeling of the patient's airways and the position of the navigation catheter 102 within the patient.

The computer system 122 may be any suitable computer system including a processor 204 and storage medium, e.g., memory 202, such that the processor 204 can execute instructions stored on the storage medium. The computer system 122 may further include a database configured to store patient data, CT data sets including CT images, CBCT images and data sets, fluoroscopic data sets including fluoroscopic images and video, 3D reconstructions, navigation plans, and any other such data. Although not explicitly illustrated, the computer system 122 may include inputs, or may otherwise be configured to receive, CT data sets, CBCT or fluoroscopic images or video, and other suitable imaging data. Additionally, the computer system 122 includes a display configured to display graphical user interfaces. The computer system 122 may be connected to one or more networks through which one or more databases may be accessed by the computer system 122.

With respect to the navigation phase, a six degrees-of-freedom electromagnetic locating or tracking system 115, or other suitable system for determining position and orientation of a distal portion of the navigation catheter 102 (e.g., Fiber-Bragg flex sensors) and/or the camera and sensor tool 101e, is utilized for performing registration of pre-procedure images (e.g., a CT image data set and 3D models derived therefrom) and the pathway for navigation with the patient as they are located on the operating table 112.

In an EMN-type system, the tracking system 115 may include the tracking module 116, the PST 118, and the location board 120 (including the markers). The tracking system 115 may be configured for use with a locatable guide (particularly the LG sensor) and/or a camera and sensor tool (particularly the sensor coils). As described above, the medical tools, e.g., the locatable guide (LG) 101a with the LG sensor 104a and/or the camera and sensor tool 101e may be configured for insertion through the navigation catheter 102 into patient P's airways (either with or without the bronchoscope 108) and are selectively lockable relative to one another via a locking mechanism, e.g., the bronchoscope adapter 109.

The location board 120 is positioned beneath patient P. The location board 120 generates an electromagnetic field around at least a portion of the patient P within which the position of the LG sensor 104a, the navigation catheter sensor 104b, the PST 118, and the distal portion of the camera and sensor tool 101e can be determined through use of a tracking module 116. An additional electromagnetic sensor 126 may also be incorporated into the end of the navigation catheter 102. The additional electromagnetic sensor 126 may be a five degree-of-freedom sensor or a six degree-of-freedom sensor. One or more of the reference sensors of the PST 118 are attached to the chest of the patient P.

Registration refers to a method of correlating the coordinate systems of the pre-procedure images, and particularly a 3D model derived therefrom, with the patient P's airways as, for example, observed through the bronchoscope 108 and allow for the navigation to be undertaken with accurate knowledge of the location of the LG sensor within the patient and an accurate depiction of that position in the 3D model. Registration may be performed by moving the LG sensor through the airways of the patient P. More specifically, data pertaining to locations of the LG sensor, while the locatable guide is moving through the airways, is recorded using the location board 120, the PST 118, and the tracking system 115. A shape resulting from this location data is compared to an interior geometry of passages of the 3D model generated in the planning phase, and a location correlation between the shape and the 3D model based on the comparison is determined, e.g., utilizing the software on the computer system 122.

The software may align, or register, a point cloud volume generated from stereographic images acquired by the camera and sensor tool 101e or an image representing a location of LG sensor with the 3D model and/or two-dimensional images generated from the three-dimension model. Alternatively, a manual registration technique may be employed by navigating the bronchoscope 108 with the LG sensor to pre-specified locations in the lungs of the patient P, and manually correlating the images from the bronchoscope 108 to the model data of the 3D model.

Though described herein with respect to EMN systems using EM sensors, the instant disclosure is not so limited and may be used in conjunction with flexible sensor, shape sensors such as Fiber-Bragg gratings, ultrasonic sensors, or any other suitable sensor that does not emit harmful radiation. Additionally, the methods described herein may be used in conjunction with robotic systems such that robotic actuators drive the navigation catheter 102 or bronchoscope 108 proximate the target.

At any point during the navigation process, tools such as a locatable guide 101a, a therapy tool (e.g., a microwave ablation tool 101b or a forceps 101d), a biopsy tool (e.g., a biopsy needle 101c), may be inserted into and fixed in place relative to the navigation catheter 102 to place one of the tools 101b-101e proximate the target or a desired location using position information from the navigation catheter 102. The position information from the sensors 104b and/or 126 of the navigation catheter 102 may be used to calculate the position of the distal tip or distal end portion of any of the tools 101b-101d.

To ensure the accuracy of the position calculations, the tools 101a-101e may be designed to extend a predetermined distance from the distal end of the navigation catheter 102 and at least the distal portions of the tools 101a-101e that extend from the navigation catheter 102 are designed to be rigid or substantially rigid. The predetermined distance may be different depending on one or more of the design of the tools 101a-101c, the stiffnesses of the tools 101a-101e, or how each of the tools 101a-101e interact with different types of tissue. The tools 101a-101e may be designed or characterized to set the predetermined distance to ensure deflection is managed (e.g., minimized) so that the virtual tools and environment displayed to a clinician are an accurate representation of the actual clinical tools and environment.

Calculating the position of the distal end portion of any of the tools 101a-101e may include distally projecting the position information from the sensors 104b and/or 126 according to tool information. The tool information may include one or more of the shape of the tool, the type of tool, the stiffness of the tool, the type or characteristics of the tissue to be treated by the tool, or the dimensions of the tool.

With respect to the planning phase, the computer system 122, or a separate computer system (not shown), utilizes previously acquired CT image data for generating and viewing a 3D model or rendering of patient P's airways, enables the identification of a target (automatically, semi-automatically, or manually), and allows for determining a pathway through patient P's airways to tissue located at and around the target. More specifically, CT images acquired from CT scans are processed and assembled into a 3D CT volume, which is then utilized to generate a 3D model of patient P's airways. The 3D model may be displayed on a display associated with the computer system 122, or in any other suitable fashion.

Using the computer system 122, various views of the 3D model or enhanced two-dimensional images generated from the 3D model are presented. The enhanced two-dimensional images may possess some 3D capabilities because they are generated from 3D data. The 3D model may be manipulated to facilitate identification of target on the 3D model or two-dimensional images, and selection of a suitable pathway through patient P's airways to access tissue located at the target can be made. Once selected, the pathway plan, the 3D model, and the images derived therefrom, can be saved, and exported to a navigation system for use during the navigation phase(s). The ILLUMISITE software suite currently sold by Medtronic PLC includes one such planning software.

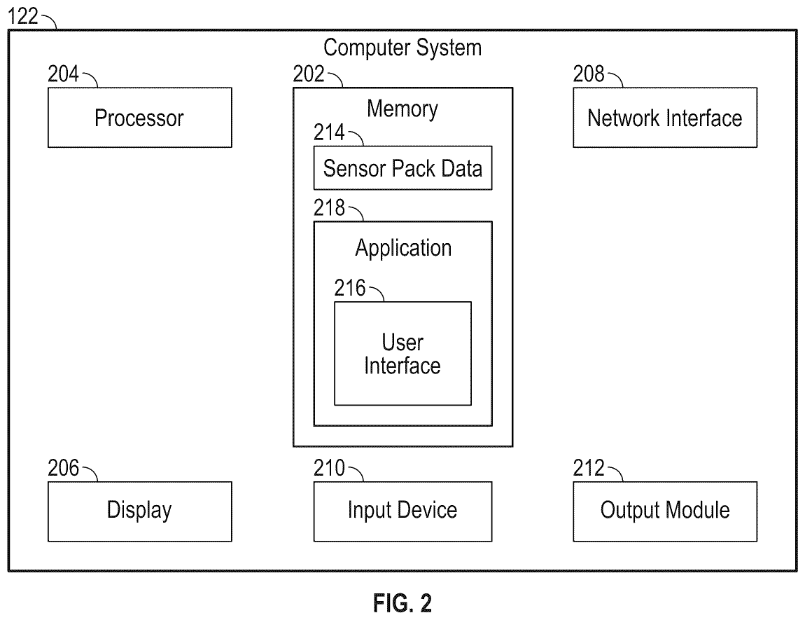

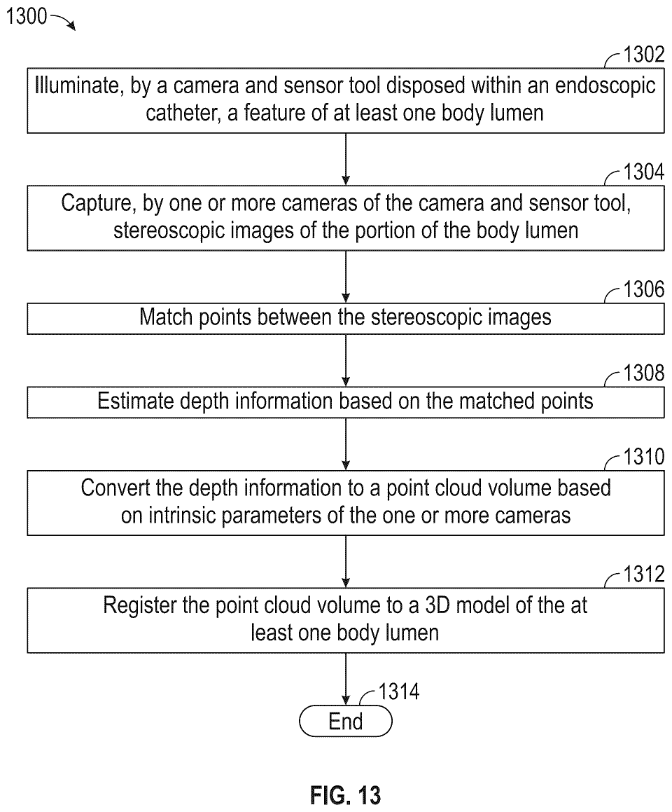

Reference is now made to FIG. 2, which is a schematic diagram of the computer system 122 of FIG. 1 configured for implementing the methods of the disclosure including the methods of FIG. 2. The computer system 122 may include a workstation. In some aspects, the computer system 122 may be coupled with the imaging system, directly or indirectly, e.g., by wireless communication. The computer system 122 may include a memory 202, a processor 204, a display 206 and an input device 210. The processor 204 may include one or more hardware processors. The computer system 122 may optionally include an output module 212 and a network interface 208. The memory 202 may store an application 218 and sensor pack data 214 including data from the one or more EM sensors 104, 126 disposed at the distal portion of the navigation catheter 102, the EM sensors of the PST 118, and data from the sensor pack 113 of the camera and sensor tool 101e. The application 218 may include instructions executable by the processor 204 for executing the methods of the disclosure including the method of FIG. 13.

The application 218 may further include a user interface 216. The image data may include preoperative CT image data, intraoperative 3D fluoroscopic image data, preoperative or intraoperative CBCT image data, and/or 3D reconstruction data. The processor 204 may be coupled with the memory 202, the display 206, the input device 210, the output module 212, the network interface 208, and the imaging system. The computer system 122 may be a stationary computer system, such as a personal computer, or a portable computer system such as a tablet computer. The computer system 122 may embed multiple computers.

The memory 202 may include any non-transitory computer-readable storage media for storing data and/or software including instructions that are executable by the processor 204 and which control the operation of the computer system 122, process data from one or more EM sensors 104, 126 disposed in or on the navigation catheter 102, e.g., at a distal end portion of the navigation catheter 102, to track the position of the navigation catheter 102 and calculate or project the position of a distal end portion of a medical tool at a fixed position within the navigation catheter 102, and, in some aspects, may also control the operation of the imaging system. The imaging system may be used to capture a series of preoperative CT images of a portion of a patient's body, e.g., the lungs, as the portion of the patient's body moves, e.g., as the lungs move during a respiratory cycle.

Optionally, the imaging system may include a CBCT imaging system or a 3D fluoroscopic imaging system that captures a series of images based on which a 3D reconstruction is generated and/or to capture a live 2D view to confirm placement of the navigation catheter 102 and/or the medical tool, e.g., the camera and sensor tool 101c. In one aspect, the memory 202 may include one or more storage devices such as solid-state storage devices, e.g., flash memory chips. Alternatively, or in addition to the one or more solid-state storage devices, the memory 202 may include one or more mass storage devices connected to the processor 204 through a mass storage controller (not shown) and a communications bus (not shown).

Although the description of computer-readable media contained herein refers to solid-state storage, it should be appreciated by those skilled in the art that computer-readable storage media can be any available media that can be accessed by the processor 204. That is, computer readable storage media may include non-transitory, volatile and non-volatile, removable and non-removable media implemented in any method or technology for storage of information such as computer-readable instructions, data structures, program modules or other data. For example, computer-readable storage media may include RAM, ROM, EPROM, EEPROM, flash memory or other solid-state memory technology, CD-ROM, DVD, Blu-Ray or other optical storage, magnetic cassettes, magnetic tape, magnetic disk storage or other magnetic storage devices, or any other medium which may be used to store the desired information, and which may be accessed by computer system 122.

The application 218 may, when executed by the processor 204, cause the display 206 to present the user interface 216. The user interface 216 may be configured to present to the user a single screen including a three-dimensional (3D) view of a 3D model of a target from the perspective of a tip of a medical tool, a live two-dimensional (2D) view showing the medical tool, and a target mark, which corresponds to the 3D model of the target, overlaid on the live 2D view. The user interface 216 may be further configured to display the target mark in different colors depending on whether the medical tool tip is aligned with the target in three dimensions.

The network interface 208 may be configured to connect to a network such as a local area network (LAN) consisting of a wired network and/or a wireless network, a wide area network (WAN), a wireless mobile network, a Bluetooth network, and/or the Internet. The network interface 208 may be used to connect between the computer system 122 and the imaging system 515. The network interface 208 may be also used to receive the sensor pack data 214. The input device 210 may be any device by which a user may interact with the computer system 122, such as, for example, a mouse, keyboard, foot pedal, touch screen, and/or voice interface. The output module 212 may include any connectivity port or bus, such as, for example, parallel ports, serial ports, universal serial busses (USB), or any other similar connectivity port known to those skilled in the art. From the foregoing and with reference to the various figures, those skilled in the art will appreciate that certain modifications can be made to the disclosure without departing from the scope of the disclosure.

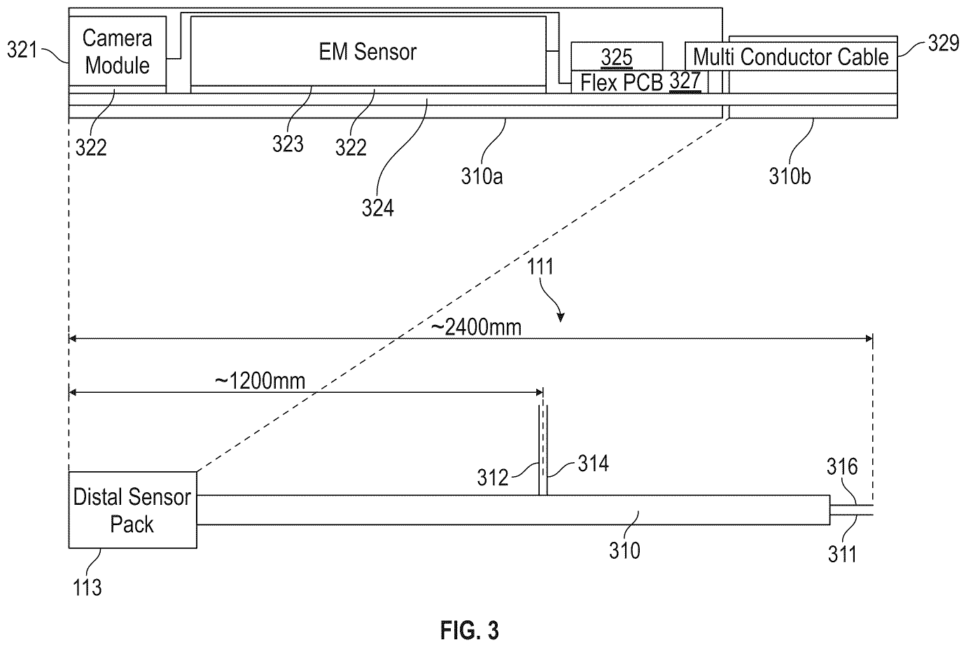

FIG. 3 is a block diagram that illustrates a camera and sensor tool 101e for use in a navigation catheter 102 or a working channel (e.g., an EWC or an sEWC 102) of an endoscope or a bronchoscope. The endoscopic or bronchoscopic camera and sensor tool 101e may include a distal sensor pack 113 and optical fibers 316 housed within a sheath 310. The sheath 310 may be a tube sheath made of, for example, Polytetrafluoroethylene (PTFE). The distal sensor pack 113 may include sensors and optical fibers 316. The distal sensor pack 113 may include a camera module 321 (which may include, for example, a miniature camera with a CMOS imaging sensor) and an electromagnetic (EM) sensor 323.

The EM sensor 323 may include multiple sensor coils, e.g., 3 to 6 sensor coils. The camera module 321 and the EM sensor 323 may be coupled to a structural wire 324 or other structure and/or material suitable for providing mechanical structure and/or stiffness. The structural wire 324 may be a rigid or semi-rigid wire, such as a metal flat wire. The camera module 321 and the EM sensor 323 may be coupled to a structural wire 324 via an adhesive 322 or any other suitable method for attaching the camera module 321 and the EM sensor 323 to the structural wire 324.

The distal sensor pack 113 may also include a flexible printed circuit board (PCB) 327, which may be mechanically coupled to the structural wire, and which may be electrically coupled to the camera module 321, which may include one or more miniature cameras, and the EM sensor 323. An inertial measurement unit (IMU) sensor 325 (e.g., an MC3672 accelerometer) may be electrically coupled to the flexible PCB 327.

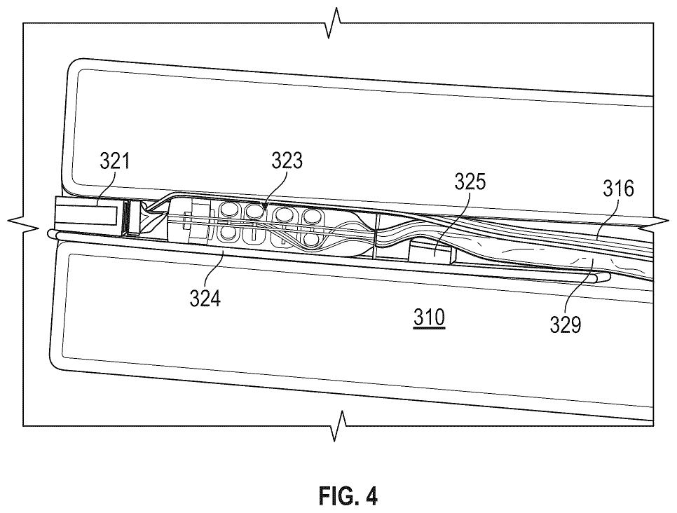

FIG. 4 is a side view of a camera and sensor tool 101e. The camera and sensor tool 101e includes a sensor pack 113 at a distal end portion of the camera and sensor tool 101c. The sensor pack 113 includes, in order proximally from a distal end portion, one or more cameras, an electromagnetic (EM) sensor assembly, and an inertial measurement unit (IMU) sensor coupled to a length of a structural wire 324. The structural wire 324 provides structure to the sensor pack 113 and may be bent at an angle or into a shape suitable for navigating a body lumen. The camera module 321 including one or more cameras, the EM sensor 323, and the IMU sensor 325 are electrically coupled to a cable assembly 329, which extends to the proximal end portion of the camera and sensor tool 101c. The sensor pack 113 and the cable assembly 329 may both be encased within a single sheath or separate sheaths 310a, 310b. In aspects, the sensor pack 113 may be encased within a sheath 310a made of a different material from the sheath 310b encasing the cable assembly 329. As illustrated in FIG. 4, the camera wire electrically coupling the camera module 321 to the cable assembly 329 may be disposed on or adjacent to the EM sensor 323 and the IMU sensor 325. In aspects, the camera wire may be disposed opposite from or nearly opposite from the structural wire 324.

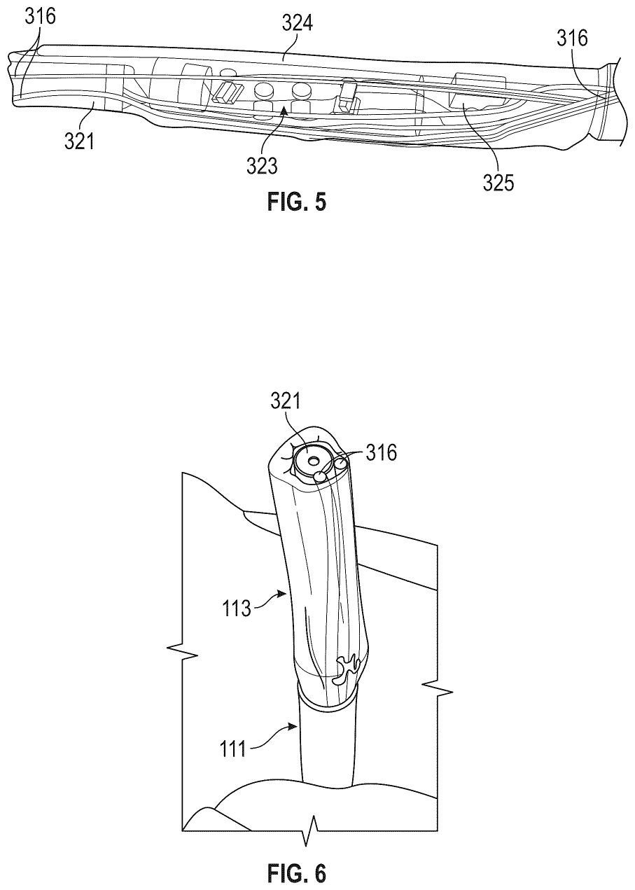

FIG. 5 is a side view of another example of a camera and sensor tool 101e. The other example of the camera and sensor tool 101e includes a sensor pack 113 at a distal end portion of the camera and sensor tool 101e. The sensor pack 113 includes, in order proximally from a distal end portion, a camera module 321, which may include one or more cameras, an electromagnetic (EM) sensor 323, and an inertial measurement unit (IMU) sensor 325 coupled to a length of structural wire 324. The structural wire 324 provides structure to the sensor pack 113 and may be bent at an angle or into a shape suitable for navigating a body lumen. The camera module 321, the EM sensor 323, and the IMU sensor 325 are electrically coupled to a cable assembly 329, e.g., a multi-conductor cable, which extends to the proximal end portion of the camera and sensor tool 101e. The sensor pack 113 and the cable assembly 329 may both be encased within a single sheath 310a, 310b. In aspects, the sensor pack 113 may be encased within a sheath 310a made of a different material from the sheath 310b encasing the cable assembly 329.

The camera module wires electrically coupling the camera module 321 to the cable assembly 329 may be disposed on or adjacent to the EM sensor 323 and the IMU sensor 325. In aspects, the camera wire 311 may be disposed opposite from or nearly opposite from the structural wire 324, e.g., a rigid or semi-rigid flat wire. The other example of the camera and sensor tool 101e also includes one or more optical fibers 316, which may be optically coupled to illumination sources of a console to which the camera and sensor tool 101e connects.

Alternatively, the one or more optical fibers 316 may be replaced with illumination sources at a distal end portion of the sensor pack 113. The illumination sources may be light emitting diodes (LEDs), which are electrically coupled to wires of the cable assembly 329. The sensor pack 113 may include a pair of illumination sources. In aspects, only a first illumination source may be activated while capturing one or more first image frames and only a second illumination source may be subsequently activated while capturing one or more second image frames. In aspects, both the first and second illumination sources may be activated while capturing one or more third image frames.

FIG. 6 is a perspective view of a distal end portion of the other example of the camera and sensor tool 101e of FIG. 5. In this aspect, the distal end portions of a single camera and illumination optical fibers 316 are exposed to enable illumination of tissue and capturing images of illuminated tissue.

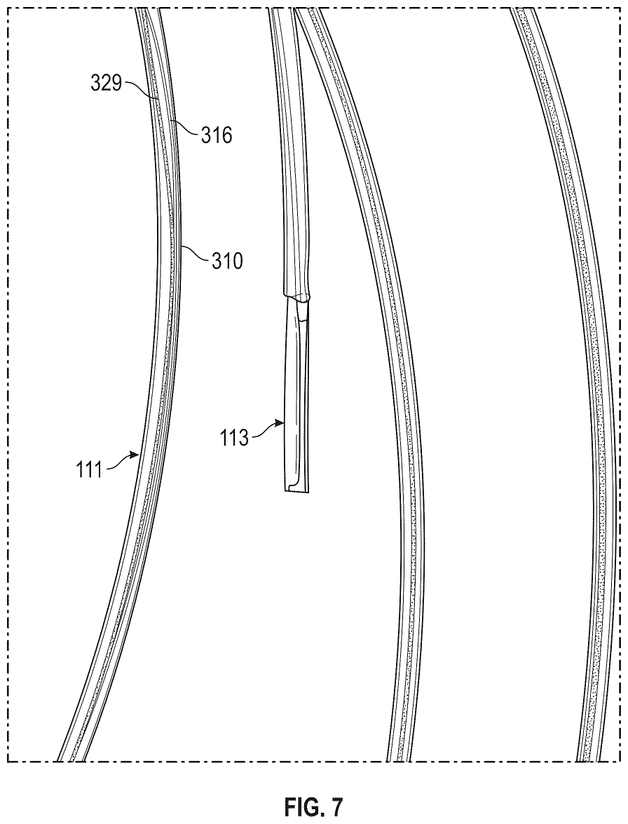

FIG. 7 is a top view of the distal end portion and length portions of the other example of the camera and sensor tool 101e of FIG. 5. As illustrated, the sheath 310a encasing the sensor pack 113 and the sheath 310b encasing the length of the cable assembly 329 may be or may approximately be the same diameter. In aspects, the cable assembly 329 is more flexible than the sensor pack 113. In aspects, the portion of the sensor pack 113 including the camera module 321, which may include multiple cameras, may be bent at an angle with respect to the remaining portion of the sensor pack 113.

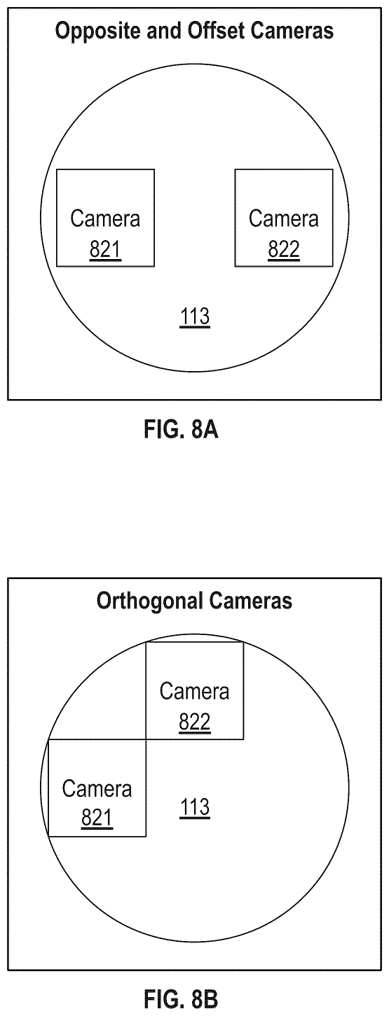

FIGS. 8A and 8B illustrate examples of dual camera configurations within examples of the camera module 321 of the sensor pack 113 of the disclosure. As illustrated in FIG. 8A, cameras 821, 822 may be arranged opposite and offset from one another. Alternatively, as illustrated in FIG. 8B, the cameras 821, 822 may be arranged orthogonal to each other. In another alternative example, the sensor pack 113 may include three cameras: A first camera may be arranged opposite and offset from a second camera, and a third camera may be arranged orthogonal to the second camera.

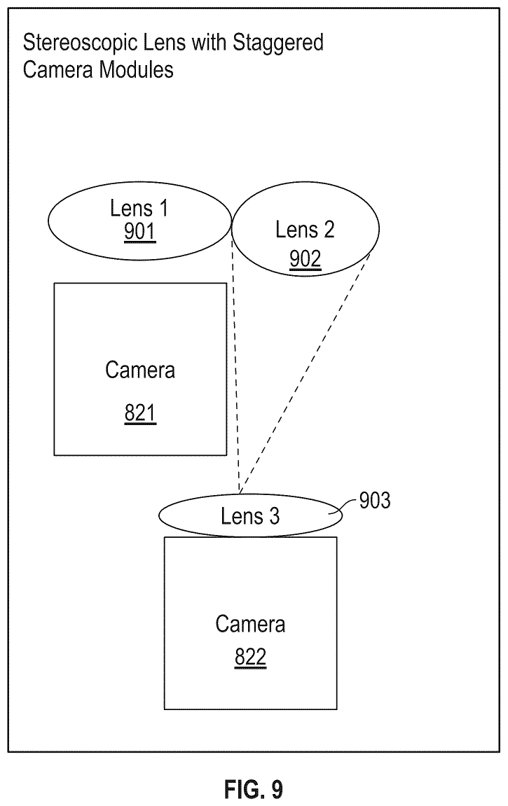

FIG. 9 is a block diagram that illustrates an example of the camera module 321 including staggered cameras 821, 822 and stereoscopic lenses 901-903. A pair of lenses 901, 902 are arranged at a distal end portion of the sensor pack 113, an aperture of a first camera 821 is arranged adjacent to a first lens 901 of the pair of lenses, and a second camera is arranged near a proximal end portion of the first camera 821 and axially offset from the first camera 821. Additionally, or alternatively, the first camera 821 and the second camera 822 are arranged in a staggered configuration. A third lens 903 may be coupled to the aperture of the second camera 822. According to this arrangement a second lens 902 of the pair of lenses focuses light passing through the second lens 902 onto the third lens 903, which transmits light to the aperture of the second camera 822. Alternatively, an optical fiber may be coupled between the second lens 902 and the third lens 903, and may be configured to transmit light (e.g., light reflected from tissue) between the second lens 902 and the third lens 903.

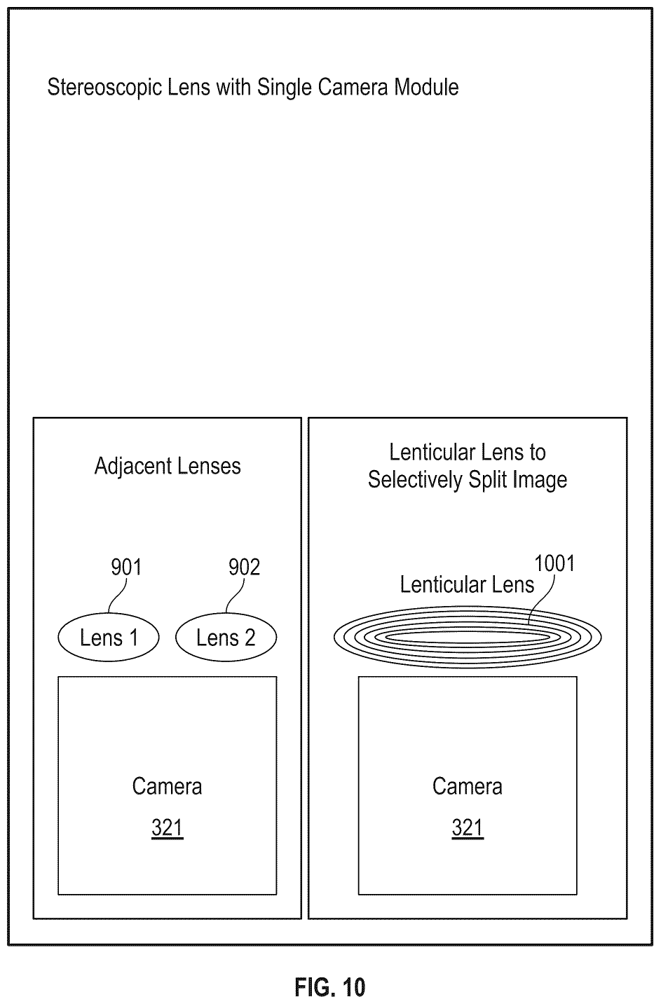

FIG. 10 shows block diagrams that illustrate examples of camera modules 321 including a single camera and stereoscopic lenses. In one example, a pair of lenses 901, 902 are arranged adjacent to each other and adjacent to the aperture of the single camera. In another example, a lenticular lens 1001 is arranged adjacent to the aperture of the single camera. The lenticular lens 1001 is configured to selectively split illumination light reflected from tissue. The lenticular lens 1001 includes a series of parallel, elongated convex lenses, which may be referred to as lenticules, on a flat substrate. The lenticules are disposed on the surface of a lenticular lens sheet. Each lenticule acts like magnifying glass, which magnifies a small portion of the image behind the lenticule. In aspects, the lenticules may be cylindrical (e.g., elongated and linear) or spherical in shape. The flat substrate provides a support structure for the lenticules and provides stability to the lenticular lens sheet. Each lenticule may display a particular portion of the image. The lenticules may be configured to detect different grades of shadows within a body lumen to acquire depth perception information used to construct a model of the body lumen. The one or more illumination sources of the sensor pack 113 and the lenticules may be configured to capture images optimized for detecting shadows on tissue and/or determining tissue depths to accurately construct a model of a portion of a body lumen.

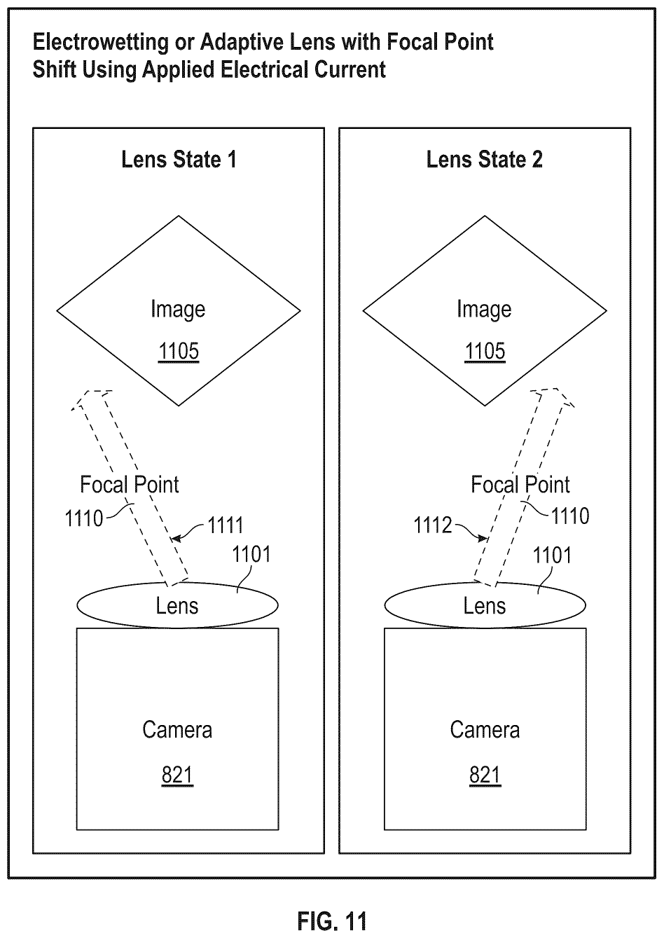

FIG. 11 shows block diagrams that illustrate an example of a camera module 321 including a single camera 821 and an adaptive lens 1101 according to an aspect of the disclosure. The adaptive lens 1101 is designed to change its focal length or focus without mechanical actuation. The adaptive lens 1101 may be designed to shift its focal point 1110 in response to an applied electrical current. For example, the adaptive lens 1101 may be an electrowetting lens. The electrowetting lens may include a chamber filled with two immiscible liquids: a conductive fluid (e.g., water) and a non-conductive fluid (e.g., an oil). The boundary between the conductive and non-conductive fluids form a dynamic lens interface. By applying a voltage to the electrowetting lens, the wettability of one fluid over a solid substrate is modified. This changes the shape of the boundary between the two fluids, which changes the lens's focal length. For example, as shown in FIG. 11, the adaptive lens 1101 may be configured to focus according to the focal point 1110 on a left portion 1111 of an image 1105 when no electrical current is applied to the adaptive lens 1101 (Lens State 1). When electrical current is applied to the adaptive lens 1101 (Lens State 2), the focal point 1110 of the adaptive lens 1101 may shift to a right portion 1112 or other desired portion of the image 1105.

In aspects, the adaptive lens 1101 may be dielectric elastomer lens. The dielectric elastomer lens may include a transparent elastomer or other suitable polymer sandwiched between compliant electrodes. The elastomer may deform in response to an electric field generated by applying an electrical voltage to the electrodes. In response to applying an electrical voltage to the electrodes, the elastomer compresses in thickness and expands in area. This deformation changes the curvature of the elastomer, and thus its optical power.

In aspects, the adaptive lens 1101 may be liquid crystal lens, which may be made from liquid crystal cells sandwiched between transparent electrodes and substrates. By applying an electrical voltage to the electrodes, the orientation of the liquid crystal molecules changes, which alters the refractive index of the liquid crystal cells. This change in refractive index effectively changes the lens's focal length. In aspects, the dual and multi-lens camera modules 321 described herein may incorporate two or more adaptive lenses 1101, including two or more electrowetting lenses, dielectric elastomer lenses, liquid crystal lenses, or any combination thereof.

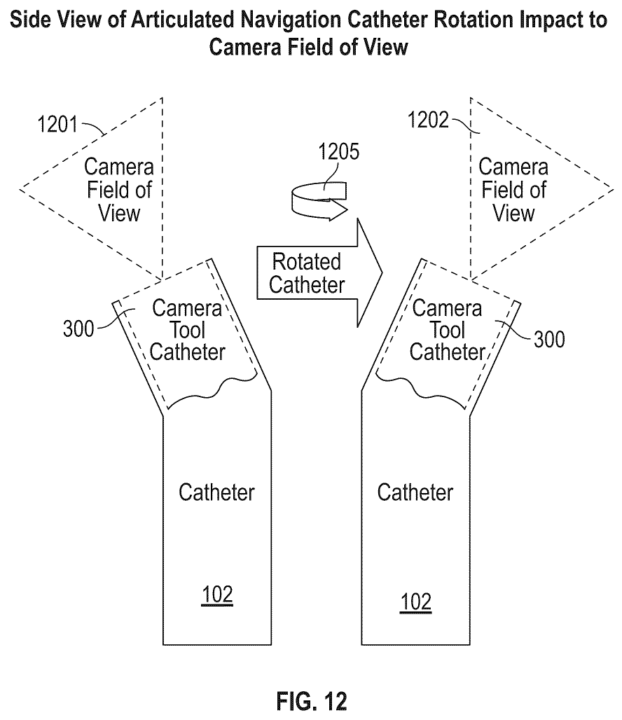

FIG. 12 is a block diagram that illustrates articulation of a camera tool to increase the field of view 1201, 1202 of the camera module 321 of the camera tool catheter 300. To achieve a greater field of view, the camera tool catheter 300 may be configured with appropriate actuation systems (e.g., pull wires) to cause a distal end portion of the camera tool (e.g., the distal sensor pack 113) to exit the distal end portion of the navigation catheter 102 at an angle. Then, while the camera module 321 captures imaging data, the navigation catheter 102 may be rotated 1205 to rotate the angled camera tool catheter 300 to capture multiple images at a fixed position. The multiple images may be stitched together to obtain a composite image with a large field of view.

In aspects, the camera and sensor tool 101e described above is navigated inside the body, e.g., the lungs, to acquire positional information and still or video images captured by the camera and sensor tool 101e. The positional information and still or video images captured by the camera and sensor tool 101e may be combined to estimate the image depth and generate a 3-dimensional point cloud of the patient anatomy. The 3D point cloud volume is generated from a known vantage point using stereoscopic images captured by the camera and sensor tool 101e using an image rectification algorithm. A machine learning algorithm may also be applied in place of, or in combination with, any of the image rectification algorithms described herein to improve computational efficiency

In general, image rectification is a process that transforms images such that pairs of images have a common image plane, making them epipolar. This is useful in stereo vision, where two cameras view a scene from different angles, and the resulting stereoscopic images need to be aligned to determine depth or disparity. The goal of an image rectification algorithm is to make the epipolar lines in both images parallel and coincident with the horizontal axis. This simplifies the search for matching points or correspondences between the two images.

The image rectification algorithm may include all or a portion of the following features or functions. In some aspects, before rectifying images, both cameras are calibrated to determine their intrinsic parameters (e.g., focal length, principal point, etc.) and extrinsic parameters (rotation, translation, etc.). The camera calibration may involve using a calibration pattern, like a chessboard pattern, and capturing multiple images of the calibration pattern from different orientations.

The image rectification algorithm may include computing a fundamental matrix (F). Using matched points between the two stereoscopic images, F is computed. This matrix encapsulates the epipolar geometry between the two views. The image rectification algorithm may also include computing homographies for rectification. For each image, a homography (e.g., a projective transformation) is computed. The homography is used to rectify the image. Computing homographies may involve decomposing F to get the epipoles (e.g., points where epipolar lines intersect) for each image and then determining the appropriate transformations to make epipolar lines parallel and horizontal.

Next, the computed homographies are applied to each image to warp each image. This results in two rectified images where corresponding points lie on the same horizontal line, thereby simplifying the matching process. Once the images are rectified, the image rectification algorithm computes the disparity, e.g., a difference in the position of a point in the left and right images. This disparity, which may be in the form of a disparity map, is directly related to the depth of the point in the real world. In aspect, the image rectification algorithm may include a post-processing stage, during which the rectified images or the disparity map are further processed. For example, the disparity maps may be filtered or refined for improved depth estimation.

Stereo image rectification simplifies the process of disparity computation. Different stereo image rectification algorithms and approaches may be used to improve accuracy, robustness, and/or computational speed. An example of a stereo image rectification algorithm is the standard epipolar rectification algorithm described above. The standard epipolar rectification algorithm is based on the geometric properties of the epipolar geometry and may use the fundamental matrix or an essential matrix. The standard epipolar rectification algorithm aligns the epipolar lines with the horizontal scan-lines of the stereoscopic images.

Another example of a stereoscopic image rectification algorithm is the Hartley's rectification algorithm. Hartley's rectification algorithm involves resampling the two stereoscopic images using a pair of 3×3 rectifying transformations to make the epipolar lines aligned and horizontal. Hartley's rectification algorithm ensures that rectified images are oriented such that they share the same row coordinates for matching points.

Another example of a stereoscopic image rectification algorithm is the polar rectification algorithm. Instead of making the epipolar lines horizontal, the polar rectification algorithm transforms the stereoscopic images into polar coordinates. The polar rectification algorithm may be advantageous in cases where the camera's epipoles are inside the image area.

Another example of a stereoscopic image rectification algorithm is a recursive rectification algorithm. The recursive rectification algorithm recursively applies rectification, thereby refining the results iteratively. The recursive rectification algorithm may provide more accurate rectification. Another example of a stereoscopic image rectification algorithm is a 3D rotation rectification algorithm, which is based on 3D rotation. The 3D rotation rectification algorithm involves rotating two stereoscopic image planes to bring them into a common plane, thereby simplifying the stereo correspondence problem. The 3D rotation rectification algorithm may be used when accurate external calibration information (e.g., information regarding rotation and translation between cameras) is available.

Another example of a stereoscopic image rectification algorithm is a non-parametric rectification algorithm. Instead of relying on camera calibration and parametric models, the non-parametric rectification algorithm uses image features and content directly to compute rectification transformations. The non-parametric rectification algorithm may be more flexible and adaptive. Another example of a stereoscopic image rectification algorithm is a shear-based rectification algorithm. The shear-based rectification algorithm may apply shearing transformations to the stereoscopic images to achieve rectification. The shear-based rectification algorithm may be viewed as a simplified version of the standard epipolar rectification algorithm. The shear-based rectification may be computationally efficient and may be used in real-time.

Another example of a stereoscopic image rectification algorithm is an automatic rectification algorithm. The automatic rectification algorithm rectifies stereo image pairs without requiring explicit calibration data. The automatic algorithm may rely on image features and content to infer the rectifying transformations. The automatic rectification algorithm may be suitable for potential scenarios where calibration data is unavailable or unreliable.

FIG. 13 is a flowchart of an example of a method 1300 of generating a point cloud volume from stereoscopic images of at least one body lumen acquired by a camera and sensor tool 101e and registering the point cloud volume to a three-dimensional (3D) model. The method 1300 may include applying a machine learning-based stereoscopic image rectification algorithm to the stereoscopic images acquired by the camera and sensor tool 101e of the disclosure. The method 1300 includes illuminating, by a camera and sensor tool 101e disposed within an endoscopic catheter, a feature of the at least one body lumen at block 1302. The camera and sensor tool 101e may be disposed within an extended working channel of the endoscopic catheter. The method 1300 also includes capturing, by one or more cameras of the camera and sensor tool 101e, stereoscopic images of the feature of the at least one body lumen at block 1304. The camera and sensor tool 101e may capture the stereoscopic images in any of a number of ways. For example, the camera may capture images while being moved, e.g., rotated, two cameras may be positioned in a compact way allowing them to fit into a very thin endoluminal medical device, and/or integrating the one or more cameras into an endoluminal medical device in a way suitable for an endoluminal procedure. At block 1306, the method 1300 includes matching points between the stereoscopic images.

At block 1310, depth information is estimated based on the matched points. Neural networks may be used to estimate depth from the stereoscopic images using stereo disparity estimation. In stereo vision, two images of the same scene are captured, e.g., by two cameras or by one camera and two lenses, from slightly different viewpoints. The two captured images, which may be referred to as left and right images, are used to compute a disparity map. The disparity map encodes the pixel-wise differences or disparities between corresponding points in the left and right images. The disparities are inversely proportional to depth: smaller disparities correspond to objects closer to a camera, while larger disparities correspond to objects farther away from the camera.

Neural networks may be employed to learn the mapping between image pairs and their associated disparities. This neural network may be referred to as a stereo matching network or stereo convolutional neural network (stereo CNN). The neural network is trained on a dataset of stereo image pairs and ground truth disparity maps. The ground truth disparity maps may be generated using precise depth measurement techniques, such as structured light. During training, the neural network minimizes a loss function that quantifies the difference between the loss function's predicted disparities and the ground truth disparities. The loss functions may include mean squared error or suitable custom loss functions designed for disparity maps.

The neural network may include convolutional layers that extract features from the left and right images independently. These features may then be fused to compute a disparity map. The neural network's final output may be a disparity map for the input stereo image pair. The disparity map may be processed to convert disparities of the disparity map to depth values of a depth map using known parameters of the camera system. Filtering and/or edge-preserving smoothing may be applied to the estimated depth map to refine the estimated depth map and remove artifacts. The estimated depth map may be used to generate a 3D reconstruction in the form, for example, of a grayscale image, in which lighter areas represent objects closer to the camera, and darker areas represent objects farther away from the camera.

The effectiveness of depth estimation using neural networks may depend on factors such as the quality of the neural network training data, the architecture of the neural network, and the choice of hyperparameters. Advanced techniques, such as attention mechanisms and multi-scale processing, may be used to improve the accuracy of depth estimation from stereo image pairs.

At block 1310, the method 1300 includes converting the depth information to a point cloud volume based on intrinsic parameters of the one or more cameras. The intrinsic parameters may include the field of view. Then, before ending at block 1314, the method 1300 registers the point cloud volume to the 3D model of the at least one body lumen, at block 1312. The 3D model may be generated based on preoperative radiographic images, such as computed tomography (CT) images. The result of registration includes the knowledge of where the camera and sensor tool 101e is located inside the bronchial tree. And with six degrees of freedom, that locational knowledge includes both position and orientation information.

Aspects of the disclosure may be applicable to a variety of medical procedures, such as electromagnetic navigation bronchoscopy procedures (ENB procedures), and any endoluminal procedure that requires registration between known patient anatomy model and a navigation platform, e.g., cardiac, GI, GYN, etc.

Various aspects disclosed herein may be combined in different combinations than the combinations specifically presented in the description and accompanying drawings. Depending on the example, certain acts or events of any of the processes or methods described herein may be performed in a different sequence, may be added, merged, or left out altogether (e.g., all described acts or events may not be necessary to carry out the techniques). In addition, while certain aspects of this disclosure are described as being performed by a single module or unit for purposes of clarity, the techniques of this disclosure may be performed by a combination of units or modules associated with, for example, a medical device.

In one or more examples, the described techniques may be implemented in hardware, software, firmware, or any combination thereof. If implemented in software, the functions may be stored as one or more instructions or code on a computer-readable medium and executed by a hardware-based processing unit. Computer-readable media may include non-transitory computer-readable media, which corresponds to a tangible medium such as data storage media (e.g., RAM, ROM, EEPROM, flash memory, or any other medium that can be used to store desired program code in the form of instructions or data structures and that can be accessed by a computer).

Instructions may be executed by one or more processors, such as one or more digital signal processors (DSPs), general purpose microprocessors, application specific integrated circuits (ASICs), field programmable logic arrays (FPGAs), or other equivalent integrated or discrete logic circuitry. Accordingly, the term “processor” as used herein may refer to any of the foregoing structure or any other physical structure suitable for implementation of the described techniques. Also, the techniques could be fully implemented in one or more circuits or logic elements.

The invention may be further described by reference to the following numbered paragraphs:
1. A camera and sensor tool comprising:
   a sensor pack at a distal end portion of the camera and sensor tool, the sensor pack including:
      a structural member;
      a cable assembly;
      one or more cameras coupled to the structural member and electrically coupled to the cable assembly;
      an electromagnetic (EM) sensor assembly coupled to the structural member and electrically coupled to the cable assembly;
      an inertial measurement unit (IMU) coupled to the structural member and electrically coupled to the cable assembly;
      an illumination source; and
      one or more lenses optically coupled to apertures of the one or more cameras.
2. The camera and sensor tool according to paragraph 1, wherein the structural member is a length of flat wire or rigid wire.
3. The camera and sensor tool according to any of the preceding paragraphs, wherein the sensor pack and the cable assembly are encased within a sheath.
4. A method of registering stereo images of at least one body lumen to a three-dimensional (3D) model, the method comprising:
   illuminating, by a camera and sensor tool disposed within an endoscopic catheter, a feature of the at least one body lumen;
   capturing, by one or more cameras of the camera and sensor tool, stereoscopic images of the feature of the at least one body lumen;
   matching points between the stereoscopic images, yielding matched points;
   estimating depth information based on the matched points;
   converting the depth information to a point cloud volume based on intrinsic parameters of the one or more cameras; and
   registering the point cloud volume to the 3D model of the at least one body lumen.
5. The method of claim 4, wherein the at least one body lumen is an airway of a lung.
6. The method according to any of the preceding paragraphs, further comprising selectively illuminating the feature of the at least one body lumen with different illumination sources of the camera and sensor tool.
7. The method according to any of the preceding paragraphs, wherein capturing the stereoscopic images of the feature of the at least one body lumen includes capturing the stereoscopic images of the feature of the at least one body lumen through a pair of lenses adjacent to an aperture of a camera of the one or more cameras.
8. The method according to any of the preceding paragraphs, wherein estimating the depth information includes estimating the depth information using a neural network.
9. The method according to any of the preceding paragraphs, further comprising generating the 3D model based on preoperative radiographic images of the at least one body lumen.
10. The method according to any of the preceding paragraphs, wherein the at least one body lumen forms at least a portion of a bronchial tree.
11. The method according to any of the preceding paragraphs, further comprising rectifying the stereoscopic images before matching points between the stereoscopic images.
12. The method according to paragraph 11, wherein rectifying the stereoscopic images includes applying an image rectification algorithm to the stereoscopic images.
13. The method according to paragraph 12, wherein the image rectification algorithm is at least one of an epipolar rectification algorithm, Hartley's rectification algorithm, a polar rectification algorithm, a recursive rectification algorithm, a 3D rotation rectification algorithm, a non-parametric rectification algorithm, a shear-based rectification algorithm, or an automatic rectification algorithm.
14. A system comprising:
   a catheter;
   a sensor pack at a distal end portion of the catheter, the sensor pack including:
      a structural member;
      a cable assembly;
      one or more cameras coupled to the structural member and electrically coupled to the cable assembly;
      an illumination source; and
      one or more lenses optically coupled to apertures of the one or more cameras;
   a processor; and
   memory having stored thereon instructions, which when executed by the processor, causes the processor to:
      illuminate, by the illumination source, a feature of the at least one body lumen;
      capture, by the one or more cameras, stereoscopic images of the feature of the at least one body lumen;
      match points between the stereoscopic images, yielding matched points;
      estimate depth information based on the matched points;
      convert the depth information to a point cloud volume based on intrinsic parameters of the one or more cameras; and
      register the point cloud volume to the 3D model of the at least one body lumen.
15. The system according to paragraph 14, wherein the structural member is a length of flat wire or rigid wire.
16. The system according to any of the preceding paragraphs, wherein the sensor pack and the cable assembly are encased within a sheath.
17. The system according to any of the preceding paragraphs, wherein the sensor pack further comprises an electromagnetic (EM) sensor assembly coupled to the structural member and electrically coupled to the cable assembly.
18. The system according to any of the preceding paragraphs, wherein the sensor pack further comprises an inertial measurement unit (IMU) coupled to the structural member and electrically coupled to the cable assembly.
19. The system according to any of the preceding paragraphs, wherein capturing the stereoscopic images of the feature of the at least one body lumen includes capturing the stereoscopic images of the feature of the at least one body lumen through a pair of lenses adjacent to an aperture of a camera of the one or more cameras.
20. The system according to any of the preceding paragraphs, wherein estimating the depth information includes estimating the depth information using a neural network.

What is claimed is:

1. A camera and sensor tool comprising:
a sensor pack at a distal end portion of the camera and sensor tool, the sensor pack including:
a structural member;
a cable assembly;
one or more cameras coupled to the structural member and electrically coupled to the cable assembly;
an electromagnetic (EM) sensor assembly coupled to the structural member and electrically coupled to the cable assembly;
an inertial measurement unit (IMU) coupled to the structural member and electrically coupled to the cable assembly;
an illumination source; and
one or more lenses optically coupled to apertures of the one or more cameras.

2. The camera and sensor tool of claim 1, wherein the structural member is a length of flat wire or rigid wire.

3. The camera and sensor tool of claim 1, wherein the sensor pack and the cable assembly are encased within a sheath.

4. A method of registering stereo images of at least one body lumen to a three-dimensional (3D) model, the method comprising:
illuminating, by a camera and sensor tool disposed within an endoscopic catheter, a feature of the at least one body lumen;
capturing, by one or more cameras of the camera and sensor tool, stereoscopic images of the feature of the at least one body lumen;
matching points between the stereoscopic images, yielding matched points;
estimating depth information based on the matched points;
converting the depth information to a point cloud volume based on intrinsic parameters of the one or more cameras; and
registering the point cloud volume to the 3D model of the at least one body lumen.

5. The method of claim 4, wherein the at least one body lumen is an airway of a lung.

6. The method of claim 4, further comprising selectively illuminating the feature of the at least one body lumen with different illumination sources of the camera and sensor tool.

7. The method of claim 4, wherein capturing the stereoscopic images of the feature of the at least one body lumen includes capturing the stereoscopic images of the feature of the at least one body lumen through a pair of lenses adjacent to an aperture of a camera of the one or more cameras.

8. The method of claim 4, wherein estimating the depth information includes estimating the depth information using a neural network.

9. The method of claim 4, further comprising generating the 3D model based on preoperative radiographic images of the at least one body lumen.

10. The method of claim 4, wherein the at least one body lumen forms at least a portion of a bronchial tree.

11. The method of claim 4, further comprising rectifying the stereoscopic images before matching points between the stereoscopic images.

12. The method of claim 11, wherein rectifying the stereoscopic images includes applying an image rectification algorithm to the stereoscopic images.

13. The method of claim 12, wherein the image rectification algorithm is at least one of an epipolar rectification algorithm, Hartley's rectification algorithm, a polar rectification algorithm, a recursive rectification algorithm, a 3D rotation rectification algorithm, a non-parametric rectification algorithm, a shear-based rectification algorithm, or an automatic rectification algorithm.

14. A system comprising:
a catheter;
a sensor pack at a distal end portion of the catheter, the sensor pack including:
a structural member;
a cable assembly;
one or more cameras coupled to the structural member and electrically coupled to the cable assembly;
an illumination source; and
one or more lenses optically coupled to apertures of the one or more cameras;
a processor; and
memory having stored thereon instructions, which when executed by the processor, causes the processor to:
illuminate, by the illumination source, a feature of at least one body lumen;
capture, by the one or more cameras, stereoscopic images of the feature of the at least one body lumen;
match points between the stereoscopic images, yielding matched points;
estimate depth information based on the matched points;
convert the depth information to a point cloud volume based on intrinsic parameters of the one or more cameras; and
register the point cloud volume to a 3D model of the at least one body lumen.

15. The system of claim 14, wherein the structural member is a length of flat wire or rigid wire.

16. The system of claim 14, wherein the sensor pack and the cable assembly are encased within a sheath.

17. The system of claim 14, wherein the sensor pack further comprises an electromagnetic (EM) sensor assembly coupled to the structural member and electrically coupled to the cable assembly.

18. The system of claim 14, wherein the sensor pack further comprises an inertial measurement unit (IMU) coupled to the structural member and electrically coupled to the cable assembly.

19. The system of claim 14, wherein capturing the stereoscopic images of the feature of the at least one body lumen includes capturing the stereoscopic images of the feature of the at least one body lumen through a pair of lenses adjacent to an aperture of a camera of the one or more cameras.

20. The system of claim 14, wherein estimating the depth information includes estimating the depth information using a neural network.
